(19) United States
(12) Patent Application Publication
(10) Pub. No.: US 2024/0393246 A1
(43) Pub. Date: Nov. 28, 2024

(54) SYSTEMS AND METHODS OF BACTERIAL GROWTH DETECTION USING DUAL LIGHT EXCITATION

(71) Applicant: BECTON, DICKINSON AND COMPANY, Franklin Lakes, NJ (US)

(72) Inventors: James R. Petisce, Westford, MA (US); Robert E. Armstrong, Hunt Valley, MD (US); David J. Turner, York, PA (US)

(21) Appl. No.: 18/796,851

(22) Filed: Aug. 7, 2024

Related U.S. Application Data
(63) Continuation of application No. PCT/US2023/062026, filed on Feb. 6, 2023.
(60) Provisional application No. 63/307,907, filed on Feb. 8, 2022.

(51) Int. Cl. G01N 21/64; C12Q 1/04; G01N 21/80

(52) U.S. Cl. CPC G01N 21/6428; C12Q 1/04; G01N 21/80; G01N 2021/6439

(57) ABSTRACT

A method of determining the presence of an analyte of interest in a blood sample within a test device including a sensor in aqueous media includes transmitting light to the test device at a first excitation wavelength at which light absorption of the sensor remains substantially constant as pH of the aqueous media changes, measuring an intensity of a first fluorescence signal emitted from the sensor in the test device in response to the first excitation wavelength, transmitting light to the test device at a second excitation wavelength, the second excitation wavelength being different from the first excitation wavelength, measuring an intensity of a second fluorescence signal emitted from the sensor in the test device in response to the second excitation wavelength, and normalizing, with the first fluorescence signal, the intensity of the second fluorescence signal emitted from the test device in response to the second excitation wavelength.

### CROSS-REFERENCE TO RELATED APPLICATIONS

This application is a continuation of PCT International Application No. PCT/US2023/062026, filed Feb. 6, 2023, which claims the benefit of U.S. Provisional Application No. 63/307,907, filed Feb. 8, 2022, each of which is hereby incorporated by reference in its entirety.

### BACKGROUND

### Field

The present disclosure relates to optimizing fluorescence detection systems, such as optical blood culture measurements systems. More particularly, the present disclosure relates to systems and methods for normalizing fluorescence signals in an optical blood culture measurement system configured to detect the presence of an analyte of interest in a sample.

### Description of the Related Art

In certain fields (e.g., medicine, pharmaceuticals, the food industry), quick and accurate determination of microorganism contamination in a particular system (e.g., a patient's blood, a batch of drug product, a food supply) is desirable. Methods employing sensors including fluorescent materials in conjunction with indicator materials have been developed for indirectly detecting microorganisms in a sample through their biological activities. Measurement systems employing these methods utilize fluorescent sensors or detectors for detecting fluorescent signals emitted from a container or vial housing the sample and sensor. Software and/or hardware systems are then utilized for processing data collected by the detectors. Signals measured by the detectors may be normalized using one or more reference signals, for example, to improve a signal-to-noise ratio in the resulting data measurements. In certain applications, an initial detector reading is taken when a vial is placed within a measurement system, and the initial reading is used as the reference signal. Normalization based on an initial detector reading may be subject to several limitations.

For example, there is often a delay between the time the sample is collected and injected into a test vial and the time the vial is placed in the measurement system. In some cases, the inoculated vial is placed within the measurement system after a number of hours or even a number of days, for example over a weekend. This means the initial detector reading may not be taken for 24-72 hours after the vial has been inoculated with the sample. The time period between when the sample is placed in the vial and when the vial is placed into the measurement instrument is commonly referred to as Delayed Vial Entry (DVE). The DVE time period may allow for the growth of bacteria or other microorganisms prior to placement of the vial within the measurement system. The growth of bacteria or other microorganisms prior to placement in the measurement system may affect the initial detector reading reference signal and, consequently, the test data normalized using the initial detector reading reference signal. Current methods for detecting DVE can employ kinetic DVE algorithms, algorithms that measure a rate of change in output signal readings over time. Such kinetic DVE algorithms also rely on an initial detector reading reference signal that can be affected by the growth of bacteria or other microorganisms prior to placement in the measurement system.

There are other drawbacks associated with using an initial detector reading as a reference signal to normalize current detector readings outputted by the measurement system. An initial detector reading reference signal may be affected by sensor temperature fluctuations. Ambient sensor temperature fluctuations can be caused by external factors such as inadequate control of the ambient environment by the end-user of the sensor and/or sensor equipment, changes in vial temperature after entry into the system, and air movement through the measurement system. Sensor temperature fluctuations may require compensation to provide accurate readings.

Current normalization techniques also fail to provide real time feedback on measurement system signal quality. The system architecture of a measurement system may cause erroneous measurements to be made. For example, some light source components for exciting fluorescent materials within the sensor may degrade in emission intensity during their useful life. The performance of optical detectors can also degrade. Changes in either the energy emission from a light source component or sensitivity of optical detectors can result in inaccurate test data being reported by the measurement system. Further sources of inaccurate data measurements may include misuse of the measurement system instrument (for example, slamming the door of the instrument enclosure). Mishandling of the measurement system can cause misalignment of a test vial in an optical interrogation path during sample testing. Additionally, inconsistencies in chemical components of the sensor within the test vial can also result in incorrect test data being reported by the measurement system.

Embodiments of the disclosed technology solve or mitigate these and other drawbacks in optical blood culture measurement systems. Implementations of the disclosed technology can address the above-described sources of variability in components of optical blood culture measurement systems. In one implementation, the component is present inside a blood culture vial received in the optical blood culture measurement system. For example, implementations of the disclosed technology can address or mitigate variability in fluorescent sensors in blood culture vials received in the optical blood culture measurement system for testing. Implementations of the disclosed technology can also address or mitigate inconsistencies associated with DVE detection. For example, implementations of the disclosed technology address or mitigate the inconsistencies in DVE detection due to the growth of bacteria or other microorganisms prior to an initial detector reading.

Embodiments of the disclosed technology are described herein with reference to optical blood culture measurement systems, such as but not limited to the BD BACTEC™ blood culture system by Becton, Dickinson and Company. It will be understood, however, that embodiments of the disclosed technology are not limited to blood culture measurement systems, and can be applied to other types of optical detection systems that rely on sensors or other materials that have a non-varying or constant characteristic suitable for use in generating a reference signal for normalization of detector readings, such as an isosbestic point. For example, embodiments of the presently-disclosed technology can be implemented in immunoassays.

### SUMMARY

Aspects of the present disclosure include systems and methods for determining the presence of an analyte of interest in a blood sample within a test device comprising a sensor in aqueous media.

In a first embodiment, a method of determining the presence of an analyte of interest in a blood sample within a test device including a sensor in aqueous media is provided. The method includes transmitting light to the test device at a first excitation wavelength at which light absorption of the sensor remains substantially constant as a pH of the aqueous media changes, measuring an intensity of a first fluorescence signal emitted from the sensor in the test device in response to the first excitation wavelength, transmitting light to the test device at a second excitation wavelength, the second excitation wavelength being different from the first excitation wavelength, measuring an intensity of a second fluorescence signal emitted from the sensor in the test device in response to the second excitation wavelength, and normalizing, with the first fluorescence signal, the intensity of the second fluorescence signal emitted from the test device in response to the second excitation wavelength.

A second embodiment includes the method of the first embodiment, wherein normalizing the intensity of the second fluorescence signal includes generating a ratio comparing the second fluorescence signal and the first fluorescence signal.

A third embodiment includes the method of the second embodiment, further including comparing the ratio to a threshold value.

A fourth embodiment includes the method of the third embodiment, further including determining the presence of the analyte when the ratio exceeds the threshold value.

A fifth embodiment includes the method of any of the first through fourth embodiments, wherein the first excitation wavelength is at an isosbestic point of a component of the sensor.

A sixth embodiment includes the method of the fifth embodiment, wherein the sensor includes a pH indicator and a fluorophore, and wherein the isosbestic point is an isosbestic point of the pH indicator.

A seventh embodiment includes the method of any of the first through sixth embodiments, wherein the first excitation wavelength is in the blue/cyan range.

An eighth embodiment includes the method of any of the first through seventh embodiments, wherein the first excitation wavelength is between 485 nm and 495 nm.

A ninth embodiment includes the method of any of the first through eighth embodiments, wherein the first excitation wavelength is about 490 nm.

A tenth embodiment includes the method of any of the first through ninth embodiments, wherein the second excitation wavelength is in the green range.

An eleventh embodiment includes the method of any of the first through tenth embodiments, wherein the second excitation wavelength is between 550 nm and 560 nm.

A twelfth embodiment includes the method of any of the first through eleventh embodiments, wherein the second excitation wavelength is about 555 nm.

A thirteenth embodiment includes the method of any of the first through twelfth embodiments, further including measuring a rate of change over time of a fluorescence output configured to change in response to proliferation of the analyte in the test device, and determining the presence of the analyte based on the normalized intensity of the second fluorescence signal and the measured rate of change over time of the fluorescence output configured to change in response to proliferation of the analyte in the test device.

A fourteenth embodiment includes the method of the thirteenth embodiment, wherein determining the presence of the analyte based on the normalized intensity of the second fluorescence signal and the measured rate of change over time of the fluorescence output configured to change in response to proliferation of the analyte in the test device includes performing a summation of the normalized intensity of the second fluorescence signal and the measured rate of change over time of the fluorescence output configured to change in response to proliferation of the analyte in the test device.

In a fifteenth embodiment, a system for determining the presence of an analyte of interest in a blood sample is provided. The system includes a blood culture test device including a sensor in an aqueous media and configured to receive a blood sample, a first light source, a first excitation filter, wherein the first excitation filter is configured to filter light from the first light source to provide light to the sensor at a first excitation wavelength at which light absorption of the sensor remains substantially constant as a pH of the aqueous media changes, a second light source, a second excitation filter, wherein the second excitation filter is configured to filter light from the second light source to provide light to the sensor at a second excitation wavelength, the second excitation wavelength being different from the first excitation wavelength, one or more detectors configured to measure an intensity of a first fluorescence signal emitted from the sensor in the test device in response to the first excitation wavelength and an intensity of a second fluorescence signal emitted from the sensor in the test device in response to the second excitation wavelength, and a processor configured to normalize the measurement of the intensity of the second fluorescence signal emitted from the sensor in the test device in response to the second excitation wavelength using the measurement of the intensity of the first fluorescence signal emitted from the sensor in the test device in response to the first excitation wavelength.

A sixteenth embodiment includes the system of the fifteenth embodiment, wherein the processor is configured to normalize the measurement of the intensity of the second fluorescence signal by generating a ratio comparing the second fluorescence signal and the first fluorescence signal.

A seventeenth embodiment includes the system of the sixteenth embodiment, wherein the processor is further configured to compare the ratio to a threshold value.

An eighteenth embodiment includes the system of the seventeenth embodiment, wherein the processor is further configured to determine the presence of the analyte when the ratio exceeds the threshold value.

A nineteenth embodiment includes the system of any of the fifteenth through eighteenth embodiments, wherein the first excitation wavelength is at an isosbestic point of a component of the sensor.

A twentieth embodiment includes the system of the nineteenth embodiment, wherein the sensor includes a pH indicator and a fluorophore, wherein the isosbestic point is an isosbestic point of the pH indicator.

A twenty-first embodiment includes the system of any of the fifteenth through twentieth embodiments, wherein the first light source includes a blue or cyan LED light source.

A twenty-second embodiment includes the system of any of the fifteenth through twenty-first embodiments, wherein the first excitation wavelength is between 485 nm and 495 nm.

A twenty-third embodiment includes the system of any of the fifteenth through twenty-second embodiments, wherein the first excitation wavelength is about 490 nm.

A twenty-fourth embodiment includes the system of any of the fifteenth through twenty-third embodiments, wherein the second light source includes a green LED light source.

A twenty-fifth embodiment includes the system of any of the fifteenth through twenty-fourth embodiments, wherein the second excitation wavelength is between 550 nm and 560 nm.

A twenty-sixth embodiment includes the system of any of the fifteenth through twenty-fifth embodiments, wherein the second excitation wavelength is about 555 nm.

A twenty-seventh embodiment includes the system of any of the fifteenth through twenty-sixth embodiments, wherein the one or more detectors are configured to measure a rate of change over time of a fluorescence output configured to change in response to proliferation of the analyte in the test device, and wherein the processor is configured to determine the presence of the analyte based on the normalized measurement of the intensity of the second fluorescence signal and the measured rate of change over time of the fluorescence output configured to change in response to proliferation of the analyte in the test device.

A twenty-eighth embodiment includes the system of any of the fifteenth through twenty-seventh embodiments, wherein in the processor is configured to determine the presence of the analyte based on a summation of the normalized measurement of the intensity of the second fluorescence signal and the measured rate of change over time of the fluorescence output configured to change in response to proliferation of the analyte in the test device.

### BRIEF DESCRIPTION OF THE DRAWINGS

FIG. 1 depicts a schematic view of a sample measurement system in accordance with an illustrative embodiment of the present disclosure.

FIG. 2 depicts a graph showing the absorption spectra of a pH indicator at various pH states in accordance with an illustrative embodiment of the present disclosure.

FIG. 3A depicts a graph showing the spectra of various optical components of a sample measurement system and the absorption spectra of a pH indicator at various pH states in accordance with an illustrative embodiment of the present disclosure.

FIG. 3B depicts a schematic view of a sample measurement system in accordance with an illustrative embodiment of the present disclosure.

FIG. 4 depicts a flowchart showing a process for optimizing detection of optical signals indicating the presence of an analyte of interest in a blood sample in accordance with an illustrative embodiment of the present disclosure.

FIG. 5 depicts a graph showing results of an experiment for detection of optical signals indicating the presence of an analyte of interest in a blood sample in accordance with an illustrative embodiment of the present disclosure.

### DETAILED DESCRIPTION

Any feature or combination of features described herein are included within the scope of the present disclosure provided that the features included in any such combination are not mutually inconsistent as will be apparent from the context, this description, and the knowledge of one skilled in the art. In addition, any feature or combination of features may be specifically excluded from any embodiment of the present disclosure. For purposes of summarizing the present disclosure, certain aspects, advantages, and novel features of the present disclosure are described herein. Of course, it is to be understood that not necessarily all such aspects, advantages, or features will be present in any particular embodiment of the present disclosure.

It is to be understood that embodiments presented herein are by way of example and not by way of limitation. The intent of the following detailed description, although discussing exemplary embodiments, is to be construed to cover all modifications, alternatives, and equivalents of the embodiments as may fall within the spirit and scope of the present disclosure.

Embodiments described herein relate to systems and methods for optimizing detection of optical signals indicating the presence of an analyte of interest in a sample, such as a blood sample. In certain embodiments, the presence of an analyte of interest, such as a bacteria or other microorganism, is determined based on detection of a change in optical properties, such as fluorescence, correlated to growth of the analyte within the sample. For example, the sample may be introduced into a test device, such as a vial, cartridge, or other container, housing a fluorescent material and an indicator material including one or more dyes that undergoes an optically-measurable change (e.g., fluorescence intensity) in response to growth of the analyte within the test device. The indicator material can be configured to change optically in response to a change in a condition in the test device or a characteristic of the sample, such as but not limited to a pH indicator that undergoes an optically-measurable change (such as a change in absorbance) in response to a change in a pH condition in the test device. The optical change of the indicator material can change the fluorescence behavior of the fluorescent material, for example, by modulating excitation of the fluorescent material and/or emission of a fluorescent signal. In certain embodiments, the fluorescent material and indicator material can be part of a sensor within the test device.

One or more detectors can detect an intensity of the fluorescent signals emitted by the fluorescent material within the test device. The data detected by the detectors can be processed to indirectly determine the presence of an analyte of interest in the test device above a threshold value, by determining a change in the intensity of the fluorescent signals emitted by the fluorescent material in response to different excitation wavelengths.

In some implementations, embodiments of the systems and methods described herein can account for or mitigate inconsistencies due to growth of bacteria or other microorganisms prior to an initial detector reading. For example, implementations of the disclosed technology detect the presence of an analyte of interest in the test device, such as a vial, cartridge or other container, above a threshold value using a reference signal based on a non-varying or constant characteristic of a component present in the measurement system, such as a non-varying or constant characteristic of the sensor in the test device. For example, the reference signal may not be dependent on a pH within a test device inoculated with a test sample. In one non-limiting example described below, the reference signal is at an isosbestic point of a component of a sensor present in the test device. The component can be a fluorescent material or an indicator material of the sensor. In some embodiments, the reference signal is a first fluorescence signal measured after excitation of the sensor using a first excitation wavelength at an isosbestic point of a component of the sensor, such as the indicator material. In some embodiments, a second fluorescence signal measured after excitation of the sensor using a second wavelength different than the first wavelength can be compared to the first fluorescence signal to detect the presence of an analyte of interest in the test device above a threshold value. In some examples, the first excitation wavelength is in the blue/cyan range. Some implementations of the disclosed technology can improve detection of DVE in optical blood culture measurement systems by detecting the presence of an analyte of interest in the test device above a threshold value using a reference signal based on a non-varying or constant characteristic of a component present in the measurement system.

Further, embodiments of the systems and methods described herein can account for or mitigate variability in components of optical blood culture measurement systems. Implementations of the disclosed technology can address the above-described sources of variability in components of optical blood culture measurement systems. In one implementation, the component is present inside a test device, such as a blood culture vial, present in the optical blood culture measurement system. For example, embodiments of the disclosed technology can address or mitigate variability in fluorescent sensors in blood culture test devices received in the optical blood culture measurement system for testing. Embodiments of the systems and methods described with reference to this implementation normalize detector readings using a reference signal that does not change as a function of the performance characteristics of the measurement system itself. Advantageously, reference signals according to the present disclosure can be based on a non-varying or constant characteristic of a component present in the measurement system, such as a non-varying or constant characteristic of the sensor in the test device. In some cases, a reference signal according to the present disclosure is not dependent on a pH of a test device inoculated with a test sample. In one non-limiting example described below, the reference signal is at an isosbestic point of a component of a sensor present in the test device. The component can be a fluorescent material or an indicator material of the sensor. In some implementations, the reference wavelength corresponds to a wavelength in the blue/cyan range, and serves as a reference channel for a second channel in the green range.

Embodiments of the present disclosure are described with reference to normalizing fluorescence signals emitted by a test device, such as a test vial, to address inconsistencies caused by the presence of an analyte prior to an initial detector reading. The embodiments of the present disclosure can also address variability in components of optical blood culture measurement systems. It will be understood that methods of adjusting, dividing, and comparing fluorescence signals using reference signals and reference ratios described herein can also be described as calibrating the fluorescence signals.

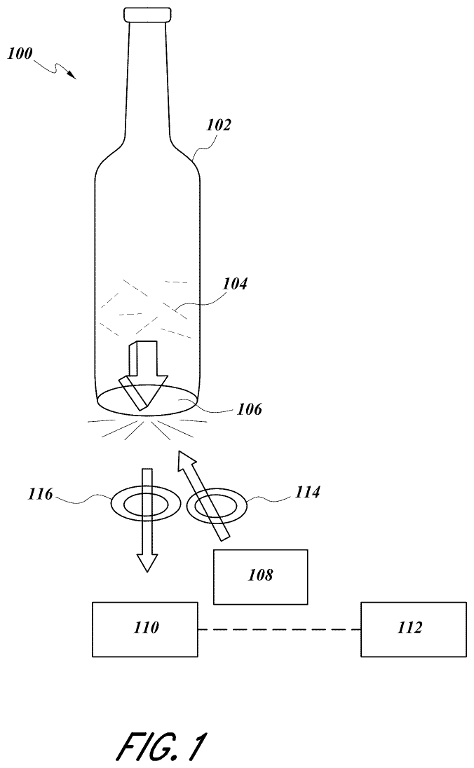

Implementations of the present disclosure that use a reference signal to normalize fluorescence signals emitted by a test device, such as a test vial, to address inconsistencies caused by the presence of an analyte prior to an initial detector reading will now be described with reference to FIGS. 1 through 5. The reference signal does not change as a function of the amount of analyte present in the test device, and can be used to normalize fluorescence signals emitted from a test vial inoculated with a sample under test. While the embodiments described with reference to FIGS. 1-5 describe a test vial, it will be understood that the embodiments of the present disclosure are not limited to systems and methods that use a test vial as a test device. In other non-limiting embodiments of the present disclosure, other test devices, such test cartridges or other containers, may be used.

FIG. 1 shows a schematic view of a measurement system 100 in accordance with an illustrative embodiment of the present disclosure. The measurement system 100 includes a test device in the form of a test vial 102, a light source 108, and a detector 110.

The test vial 102 is configured to receive a sample 104, such as a blood sample. The measurement system 100 is configured to determine the presence or absence of an analyte of interest in the sample 104 received in the test vial 102. The analyte of interest can be, for example, a microorganism or bacteria. The test vial 102 can further house a sensor 106 including a fluorescent material and an indicator material. The vial 102 may also house liquid media, which may support the growth of microorganisms within the vial 102. The test vial 102 can be, for example, a blood culture bottle.

The light source 108 can be activated to emit light at one or more wavelengths or ranges of wavelengths to excite the fluorescent material of the sensor 106. In certain embodiments, the light source 108 can include one or more light-emitting diodes (LEDs).

The detector 110 can be configured to detect fluorescence emitted by the fluorescent material of the sensor 106 following excitation thereof. The detector 110 can be a silicon photodiode, a PIN silicon diode, a GaAsP photodiode or any other suitable photodetector. In some embodiments, the detector 110 can include a photovoltaic device, a photoresistive device, a photoconductive device, or any other suitable device for detecting a signal emitted from the sensor 106. In certain embodiments, a plurality of detectors 110 may be employed for measuring fluorescence signals emitted by the sensor 106.

The system 100 may include one or more excitation filters 114 configured to filter light from the light source 108 to provide only light of a particular wavelength or range of wavelengths to the fluorescent material. For example, in certain embodiments, one or more excitation filters 114 may filter light to provide a particular wavelength or range of wavelengths to the fluorescent material that correspond to an absorption spectrum of the fluorescent material.

In certain embodiments, the system 100 can be configured to provide light at a plurality of excitation wavelengths or ranges of wavelengths. For example, the light source 108 may emit light at a plurality of emission spectra. For example, the light source 108 may include a plurality of LEDs that emit light at different emission spectra. The system 100 can include a plurality of filters 114 configured to filter light from the plurality of LEDs to provide only light of a particular wavelength or range of wavelengths to the fluorescent material from each LED. Alternatively, the light source 108 may include a single LED that can be filtered by a plurality of excitation filters 114 to provide a plurality of excitation wavelengths or ranges of wavelengths to the sensor.

The system 100 may include one or more emission filters 116 configured to filter light to provide a wavelength or range of wavelengths to the detector 110. For example, in certain embodiments, one or more emission filters 116 may filter light to provide a wavelength or range of wavelengths to the detector 110 that correspond to an emission spectrum of the fluorescent material.

The fluorescent material used in the system 100 may be selected based on the emission spectrum of the light source 108 and/or the specifications of the detector 110. In certain embodiments, the fluorescent material can include one or more fluorophores. Examples of fluorophores that may be suitable for use with the embodiments described herein include, but are not limited to, Thionin, Naphtho fluorescein, Carboxynaptho fluorescein, 3,3′-dimethyloxadicarbocyanine, Sulforhodamine B, Pyronine B, Rhodamine B, Nile red phenoxazon 9, Evans blue, Rhodamine 6G perchlorate, Sulforhodamine G, 7-aminoactinomycon D, EOSIN, Rhodamine 110, and Rhodamine 123.

As described herein, the indicator material within the sensor 106 may undergo an optical change in response to a change in an analyte of interest within the sample, for example a change in the concentration of the analyte within the sample. In certain embodiments, an indicator material is selected that undergoes changes in optical properties based on changes in the concentration of one or more of CO₂, O₂, H₂S, NH₃, or any other suitable compound known in the art, present in the test vial. In certain embodiments, an indicator is selected that undergoes changes in optical properties based on changes in pH in the test vial, where the changes in pH are due to changes in concentration of the analyte in the sample.

In certain embodiments, the indicator material can include a pH indicator. Examples of pH indicators that may be suitable for use with the embodiments described herein include, but are not limited to, Propyl Red, P-nitrophenol, Azolitmin, Chlorophenol red, 3,6-dihydroxy xanthone, Alizarin, Bromxylenol blue, M-dinitrobenzoylencurea, Bromthymol blue, Aurin (Aosolic acid), Neutral red, Cresol red, Bromocresol red, Bromocresol purple, Resolic acid, Nile Blue, Phenol red, Nitramine, Cresol purple, and Methyl yellow.

The optical change in the indicator material can act as an optical filter to change the amount of light exciting the fluorescent material or emitted from the fluorescent material of the sensor 106. Accordingly, a change in the concentration of the analyte of interest within the sample can cause a change in the fluorescent signal detected by the detector 110 by changing the optical properties of the indicator material of the sensor 106. Consequently, changes in the intensity of the fluorescent signals detected by the detector 110 may be indicative of a change in the concentration of the analyte of interest within the sample.

As an example, in certain embodiments, the system 100 is configured to detect the presence of bacteria or microorganisms within a sample placed within the vial 102. In embodiments in which bacteria is the analyte of interest, the indicator may be a pH indicator, which is configured to undergo a change in absorbance as the pH changes. When bacteria grow, carbon dioxide (CO₂) is respired. Carbon dioxide can mix with aqueous media within the vial 102 to produce carbonic acid. Increased amounts of carbonic acid result in a decrease in pH. At certain excitation wavelengths, the absorbance of the pH indicator is reduced as the pH within the vial 102 decreases, which allows for more excitation energy to reach the fluorescent material within the sensor 106, resulting in an increase in the intensity of fluorescent emission from the fluorescent material. As described herein, the fluorescent material can include one or more fluorophores. The detector 110 can detect the increased fluorescent emission intensity, which may act as an indirect measurement of an increase in carbon dioxide (CO₂) concentration. As described above, carbon dioxide concentration is directly correlated with bacterial growth. Accordingly, detection of an increased fluorescent intensity by the detector 110 may indicate the presence of bacteria within the sample.

In certain embodiments, the measurement system 100 may further include a processor 112 configured to perform signal processing to determine presence of the analyte based on changes in measured fluorescence intensity by the detector 110. In certain embodiments, the processor may be part of a computing system. Such a computing system may also include one or more of a memory, an input, and a display. The memory, which can include read-only memory (ROM) or both ROM and random access memory (RAM), can be configured to provide instructions and data to the processor 112. For example, the memory can store one or more modules that store data values defining instructions to configure processor 112 to perform signal processing functions.

In certain embodiments, the fluorescence signals detected by the one or more detectors may be normalized by the processor 112 using a reference signal.

In certain embodiments, the system 100 can be a BD BACTEC™ blood culture system by Becton, Dickinson and Company. A BACTEC™ sepsis test relies upon a silicone matrix-based sensor including an indicator compound that has pH dependent UV absorption properties and a fluorescent dye system. If bacteria are present in a blood test specimen, their growth causes the production of carbon dioxide that is converted to carbonic acid upon dissolution into the BACTEC™ media solution. In use, incident light from an LED light source positioned beneath a BACTEC™ sensor bottle is modulated in intensity by the pH dependent indicator compound within the silicone matrix-based sensor. As this incident light is modulated in intensity, the amount of incident light reaching the fluorescent dye system is also modulated resulting in a change in fluorescent light emission intensity from the fluorescent dye system.

In current use of the BACTEC™ test measurement system, an output of the measurement system at any particular time is generated based on a ratio of a current detector reading to an initial detector reading taken at the time the test bottle is first placed in the system (“time zero”). In this system, the current detector reading is normalized by dividing the current detector readings by the initial detector reading at time zero. Variability of the reported measurement system reading can reduce the sensitivity of the measurement system. Consequently, detector variability may affect a threshold measurement required for determination of the presence of an analyte in a test sample. Additional performance problems within the measurement system, such as Delayed Vial Entry (DVE), variation in instrument temperature during sample measurement, and degradation of LED light sources and fluorescence detectors, are not currently accommodated. Embodiments of the present disclosure solve these and other problems.

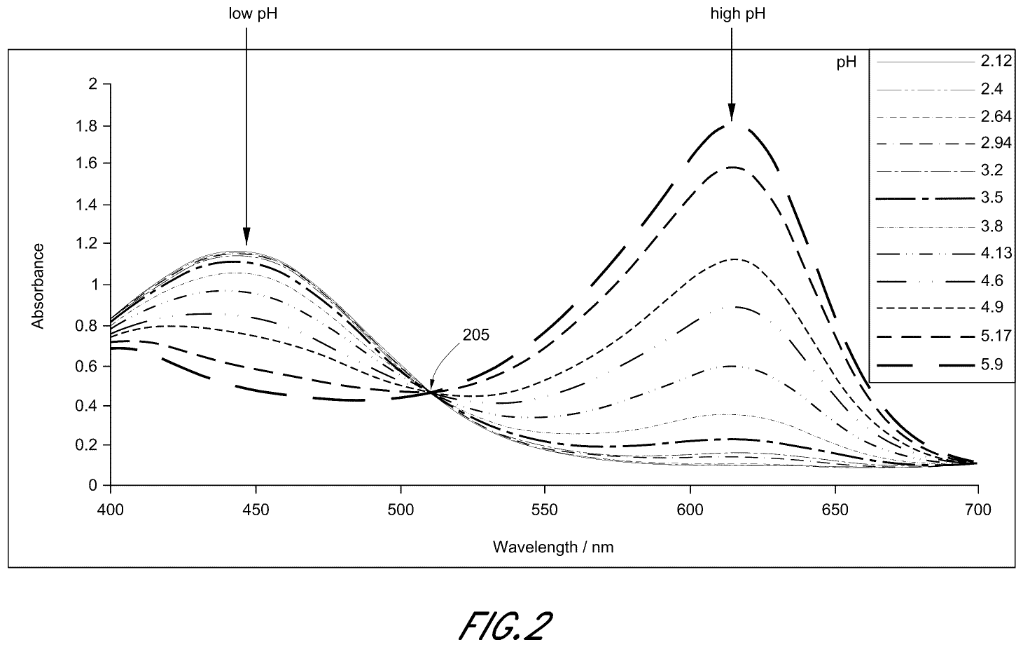

In certain embodiments, the measurement system normalizes detector readings using a reference signal that does not change as a function of the performance characteristics of the measurement system itself. For example, a reference signal can be selected that is based on a non-varying or constant characteristic of a component present in the measurement system, such as a non-varying or constant characteristic of the sensor in the test vial. In one non-limiting example of the present disclosure, a reference signal is selected that is not dependent on a pH within the test vial inoculated with a test sample. In non-limiting examples described with reference to FIGS. 2-4 below, the reference signal is at an isosbestic point of a component of a sensor present in the test vial.

An isosbestic point in this application is defined as a wavelength, wavenumber, or frequency at which a total absorbance of a sample remains constant, or substantially constant, during a chemical or a physical change of a component present in the test vial inoculated with a sample. A chemical or physical change can include, for example, a change in pH of the media in the test vial. An isosbestic point of the component present in the measurement system can be used to generate a reference signal to normalize detector readings. The component can be a pH-dependent indicator compound present in the test vial inoculated with a sample. In a non-limiting example described in detail with reference to FIG. 2 below, an isosbestic point of the indicator material of the sensor 106 may be determined and used to generate a reference signal. In cases where the indicator material is a pH indicator, an isosbestic point may be a specific wavelength where the absorption spectra of the pH indicator at various pH conditions cross. For example, as shown in FIG. 2, a pH dependent indicator compound can be present in two states within the sensor 106. A first state is present at relatively low PH levels, and a second state is present at relatively high pH levels. Each state has its own characteristic maximum absorption wavelength. These two states are in a pH dependent equilibrium with one another. Due to this pH dependent equilibrium, there exists one, non-varying or constant cross-over absorption wavelength, which is referred to herein as an isosbestic point. At the isosbestic point, the light absorption of the sensor remains substantially constant as a pH of the aqueous media changes. At the isosbestic point, the light absorption of the sensor does not vary substantially as a pH of the aqueous media changes.

The isosbestic point of the component (for example, the pH indicator) is an inherent property of the material. As a result, using fluorescence output from an excitation wavelength at the isosbestic point as a reference signal according to the present disclosure does not rely on or change with the performance characteristics of the measurement system. Advantageously, the isosbestic point of a component present in the measurement system can enable real time, continuous normalization of blood culture readings detected by the measurement system.

An isosbestic point for an indicator material of a sensor may be used to generate a reference signal for normalization of detector readings. In cases where the indicator material is a pH indicator, an isosbestic point may be a point in the absorption spectrum (i.e., specific wavelength) at which the absorption curves of the pH indicator at various pH states cross.

An example of such an isosbestic point is shown at isosbestic point 205 in FIG. 2. FIG. 2 shows a graph depicting absorption spectra for a pH indicator at various pH states including an absorption spectra at a pH of 2.12 (low pH state) and an absorption spectra at a pH of 5.9 (high pH state). As shown in FIG. 2, the absorption spectra for the pH indicator at each of the illustrated pH states overlaps at an isosbestic point 205. In this non-limiting example, the isosbestic point 205 is about 525 nm. It will be understood that the isosbestic point of a pH indicator is dependent on the concentration of the pH indicator in the sensor. The particular isosbestic point of a pH indicator provided at a particular concentration in a sensor of the test vial can be determined empirically or using any other suitable method.

Dual Excitation Light Measurement System Using Isosbestic Point of a pH Indicator Excited with Blue/Cyan Light as a Reference Signal

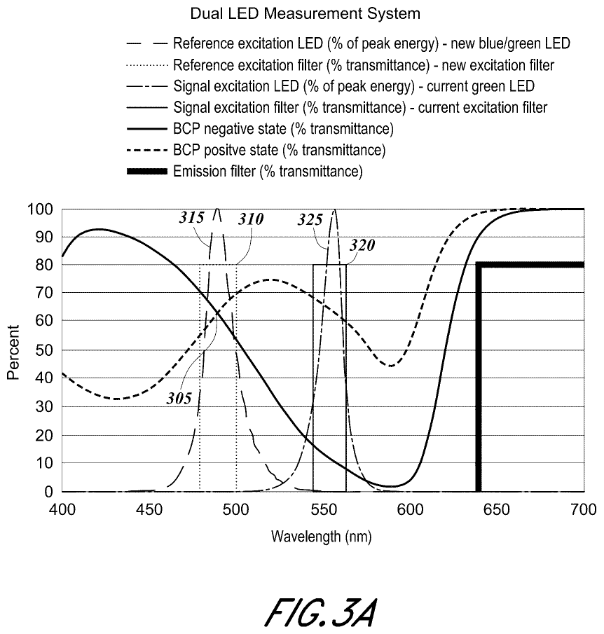

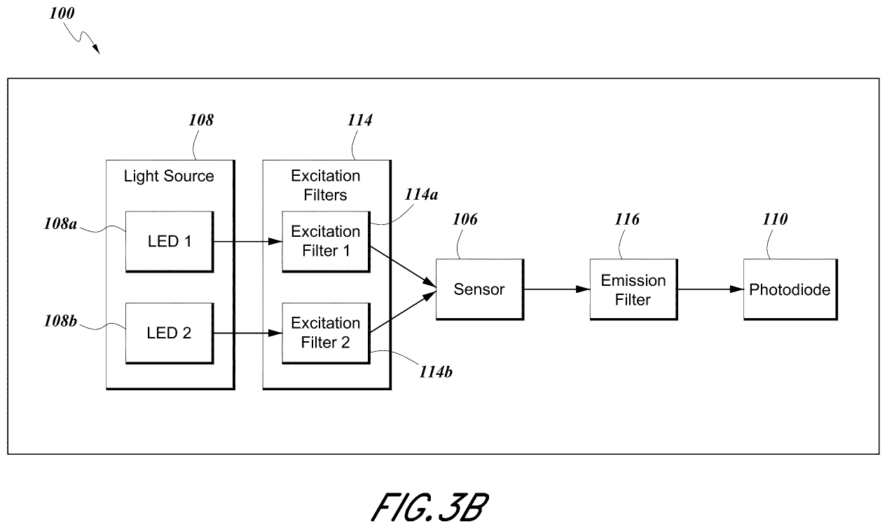

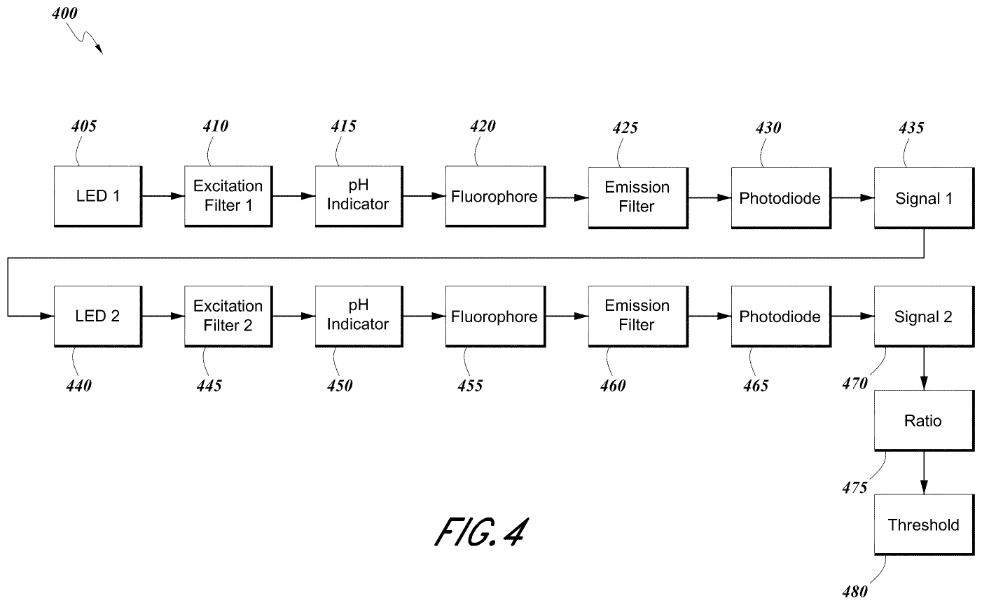

In a non-limiting embodiment described with reference to FIGS. 3A-4, a ratio of pH indicator absorbances at two specified wavelengths, wherein one of the specified wavelengths corresponds to an isosbestic point, can be used to detect the presence of an analyte, such as bacteria or other microorganisms, within a test vial.

In certain implementations, light at a first excitation wavelength or range of wavelengths at or near an isosbestic point of the pH indicator can be provided to the sample, and light at a second excitation wavelength or range of wavelengths different from the first excitation wavelength or range of wavelengths can be provided to the sample. A ratio of pH indicator absorbances at the two specified wavelengths or ranges of wavelengths can be used to determine presence of an analyte, such as bacteria or other microorganisms, within the test vial, for example, by comparison to an empirically determined threshold value.

FIG. 3A shows a graph depicting absorption spectra for an indicator material, in this case a pH indicator at a positive state (corresponding to a relatively high pH, thus indicating absence of the analyte of interest) and a negative state (corresponding to a relatively low pH, thus indicating presence of the analyte of interest). In this example, the pH indicator is Bromocresol purple. As shown in FIG. 3A, when the pH indicator is excited with light having a wavelength of about 490 nm, the absorption spectrum for the pH indicator in the positive state and the absorption spectrum for the pH indicator in the negative state overlap at an isosbestic point 305. Accordingly, in this non-limiting example, the isosbestic point 305 is about 490 nm. The absorbance of the pH indicator excited at the non-varying cross-over absorption wavelength of 490 nm remains constant, or substantially constant. In other words, the absorbance of the pH indicator does not change, or does not change substantially. Thus, the isosbestic point of the pH indicator in this example, which occurs when the pH indicator is excited using blue or cyan light at about 490 nm, is a constant that can be used to generate a reference signal (or a reference channel), as will be described in further detail below.

A wavelength of 490 nm generally corresponds to cyan light having a range of about 490 nm to about 520 nm, or blue light having a range of 490 nm to 450 nm. Although implementations of the present disclosure are described herein with reference to blue/cyan light, it will be understood that these color descriptions are not intended to be limiting. It will be understood that describing the color of light at the isosbestic point in this example as blue/cyan is for ease of reference, because light at a particular wavelength can be described as having different colors when the wavelength falls within overlapping color ranges.

FIG. 3A also depicts example spectra of optical components that may be used in a dual LED measurement system 100 according to the present disclosure. The system 100 includes a first LED light source (which emits excitation light centered around the isosbestic point of about 490 nm), a first excitation filter, a second LED light source (which emits excitation light centered around 555 nm), a second excitation filter, and an emission filter. An example of an implementation of the measurement system 100 having a first LED light source 108a, a second LED light source 108b, a first excitation filter 114a, and a second excitation filter 114b is shown in FIG. 3B.

FIG. 3A depicts an emission spectrum 315 of the first LED light source 108a (shown in FIG. 3B). A first filter window 310 filters light from the first LED light source 108a. The first filter window 310 also encompasses or is near the isosbestic point 305 of the pH indicator absorbance spectrum. Therefore, the absorbance of the pH indicator at the first excitation wavelength does not change as a function of pH. As described above, in the non-limiting example shown in FIGS. 3A and 3B, the first LED light source 108a can be a blue or cyan LED light source. In the non-limiting example shown in FIGS. 3A and 3B, the pH indicator can be Bromocresol purple. It will be understood that embodiments of the present disclosure are not limited to systems and methods that use Bromocresol purple as a pH indicator. In another non-limiting embodiment of the present disclosure, the pH indicator can be, for example, Cresol red.

FIG. 3A depicts an emission spectrum 325 of the second LED light source 108b (shown in FIG. 3B). A second filter window 320 filters the second LED light source 108b. As shown in FIG. 3A, the absorbance of the pH indicator in the second filter window 320 is significantly higher when the pH dependent indicator is in the positive state in comparison to the negative state. This is in contrast to the substantially non-varying or constant absorbance of the pH indicator at the negative state and the positive state at the isosbestic point 305 within the first filter window 310. In the non-limiting example shown in FIGS. 3A and 3B, the second LED light source 108b can be a green LED light source emitting wavelengths centered around 555 nm.

Due to the non-varying absorbance of the pH indicator at the first excitation wavelength or range of wavelengths, an emission reading from the test vial after excitation using the first excitation wavelength can act as a non-varying reference signal for comparison of an emission reading from the test vial after excitation using the second excitation wavelength or range of wavelengths. A ratio of the emission reading from the test vial after excitation using the first excitation wavelength or range of wavelengths and the emission reading from the test vial after excitation using the second excitation wavelength or range of wavelengths has been found to be proportional to the pH within the vial. This ratio is proportional to the relative slope of the absorbance spectrum between the first excitation wavelength and the second excitation wavelength.

The relationship between the emission reading at the first excitation wavelength (“1^{st}λ emission”), the emission reading at the second excitation wavelength (“2^{nd}λ emission”), and pH within the vial can be described by Equation 1:

$$\frac{{2}^{nd} λ emission}{{1}^{st} λ emission} = f ( pH ) \qquad ( Equation 1 )$$

The relative slope of the absorbance spectrum between the first excitation wavelength and the second excitation wavelength can be described by Equation 2, wherein Δ 2^{nd}λ emission is change in emission reading between a first emission reading at the second excitation wavelength and a second emission reading at the second excitation wavelength:

$$\frac{Δ {2}^{nd} λ emission}{{1}^{st} λ emission} \approx Δ slope \qquad ( Equation 2 )$$

The relative slope of the absorbance spectrum can also be described by Equation 3 below, wherein “pH1” is a first pH value at a first emission reading at the second excitation wavelength, “pH1λ emission” is the first emission reading at pH1 at the second excitation wavelength, pHn is a pH value at a time point after the first emission reading, “pHnλ emission” is the emission reading at pHn at the second excitation wavelength, “pH1slope” is the slope of the absorbance spectrum at pH1, and “pHnslope” is the slope of the absorbance spectrum at the pHn:

$$\frac{( pH 1 λ emission - pH n λ emission )}{{1}^{st} λ emission} \approx pH 1 slope - pH n slope \qquad ( Equation 3 )$$

FIG. 4 shows a flowchart depicting a process 400 for determining the presence of an analyte, such as bacteria or other microorganisms, using a measurement system similar to the measurement systems 100 described with respect to FIGS. 1 and 3B. The measurement system in this implementation is a dual light source measurement system according to the present disclosure. The measurement system includes excitation and emission filters with specific selected criteria. In accordance with implementations of the present disclosure, one of the two light sources excites a sensor with light at a first wavelength that coincides with an isosbestic point of the sensor, and the other of the two light sources excites the pH indicator with light at a second, different wavelength than the first light source. The measurement system measures emissions following excitation with the first light source, and uses these measured emissions as a reference signal or channel to assess emissions following excitation with the second light source.

Turning to FIG. 4, the process 400 includes exciting a sensor, such as sensor 106, containing a pH indicator and a fluorophore at two separate wavelengths (or ranges of wavelengths), wherein one of the wavelengths correlates to an isosbestic point of the absorbance spectra of the pH indicator.

The process 400 begins at a step 405, wherein a first light source, such as the first LED light source 108a, is activated to emit light at a first wavelength or over a first range of wavelengths to excite the fluorophore. For example, the first light source can be configured to emit light over the emission spectrum 315 depicted in FIG. 3A.

At a step 410, a first excitation filter, such as excitation filter 114a, filters the light emitted by the first light source. The excitation filter can be selected so that the filter window 310 defined by the first excitation filter encompasses or is near the isosbestic point of the pH indicator absorbance spectrum. In this non-limiting example, the isosbestic point of the pH indicator is about 490 nm. It will be understood that embodiments of the present disclosure are not limited to pH indicators having an isosbestic point of about 490 nm. The isosbestic point for a pH indicator is a property specific to that indicator and can be determined in any suitable manner, including empirically. After the first excitation filter filters the light emitted by the first light source, the pH indicator absorbs at least some of the light emitted by the first light source at a step 415. As described above, the quantity of light absorbed by the pH indicator is the same or substantially same whether the pH indicator is in a high pH state (indicating absence of the analyte of interest) or in a low pH state (indicating presence of the analyte of interest).

After the pH indicator absorbs at least some of the light emitted by the first light source, a fluorophore in the sensor absorbs at least some of the light emitted by the first light source, and in response, emits a first fluorescence signal at step 420. The first fluorescence signal emitted by the fluorophore is then filtered by an emission filter, such as emission filter 116, at a step 425.

After the first fluorescence signal emitted from the fluorophore is filtered by the emission filter, the resulting signal is detected by a photodiode, such as photodiode 110, at step 430. The photodiode can then produce an electronic signal proportional to detected light intensity at step 435. Due to the excitation of the pH indicator at a wavelength at or near the isosbestic point of the pH indicator absorbance spectrum, the resulting electronic signal “Signal 1” at step 435 can be used as a reference signal that does not change as a result of changes in pH of the pH indicator.

At a step 440, a second light source, such as second LED light source 108b, is activated to emit light at a second wavelength or over a second range of wavelengths to excite the fluorophore. For example, the second light source can be configured to emit light over the emission spectrum 325 depicted in FIG. 3A. The second wavelength or range of wavelengths is different than the first wavelength or range of wavelengths. In one example, the second light source emits light at a second wavelength that does not fall within a first range of wavelengths emitted by the first light source. In another example, the second light source emits light over a second range of wavelengths that does not overlap with a first wavelength emitted by the first light source. In still another example, the second light source emits light over a second range of wavelengths that does not overlap with a first range of wavelengths emitted by the first light source.

At a step 445, a second excitation filter, such as excitation filter 114b, having a filter window, such as filter window 320, filters the light emitted by the second light source. After the second excitation filter filters the light emitted by the second light source, the pH indicator absorbs at least some of the light emitted by the second light source at a step 450. As described above, the pH-dependent indicator compound has a single isosbestic point (a single, non-varying cross-over absorption wavelength) at which absorption by the indictor is substantially constant or does not substantially vary despite changing pH conditions. The first light source excites the pH indicator with a wavelength of light that is at or near the isosbestic point of the pH indicator, while the second light source excites the pH indictor at a different wavelength of light. Accordingly, when excited with the second light source, the pH indicator will absorb a different quantity of light when the pH indicator is in a high pH state (indicating absence of the analyte of interest) than when the pH indicator is in a low pH state (indicating presence of the analyte of interest).

After the pH indicator absorbs at least some of the light emitted by the second light source, the fluorophore in the sensor absorbs at least some of the light emitted by the second light source, and in response, emits a second fluorescence signal at step 455. The second fluorescence signal emitted by the fluorophore is then filtered by the emission filter at a step 460.

After the second fluorescence signal emitted from the fluorophore is filtered by the emission filter, the resulting signal is detected by the photodiode at step 465. The photodiode can then produce an electronic signal “Signal 2” proportional to detected light intensity at step 470.

At step 475, a ratio is generated comparing Signal 1 produced at step 435 proportional to detected light intensity at the photodiode due to excitation by the first excitation wavelength, and Signal 2 produced at step 470 proportional to the detected light intensity at the photodiode due to excitation by the second excitation wavelength. This ratio has been found to be proportional to the pH of the media in the test vial. This ratio also indicates an average slope of absorbance spectra for the pH indicator between the first excitation wavelength and the second excitation wavelength (between about 490 nm and 580 nm).

As described herein, the pH within the test vial may be correlated to the presence of an analyte, such as bacteria or other organisms, within the test vial. For example, as described above, when bacteria grow, carbon dioxide (CO₂) is respired. Carbon dioxide can mix with aqueous media within the vial 102 to produce carbonic acid. Increased amounts of carbonic acid result in a decrease in pH. Thus, the pH in the media may act as an indirect measurement of the presence of bacteria within the media. In a step 480, the ratio determined at step 475 can be compared to a predetermined threshold value to determine the presence of the analyte of interest in the test vial. In certain embodiments, the threshold value can be determined once (empirically, for example) for a particular pH indicator in a particular measurement system, such as measurement system 100. The threshold value can then be used for subsequent measurements of test vials using that particular indicator in that particular measurement system. For example, the threshold may be determined for a particular pH indicator when the measurement system is initially set up or when a new type of test vial bottle is implemented in the measurement system.

In certain implementations of the process 400 can be used to detect DVE. The process 400 can be performed when the vial is first placed in the measurement system to determine the presence of an analyte of interest in the vial at the time of placement of the vial in the measurement system. Advantageously, determining the presence of the analyte of interest above a threshold value at the time of placement of the vial in the measurement system in accordance with the present disclosure can indicate growth of bacteria or other microorganisms, prior to placement of the vial within the measurement system. Using this principle, the ratio determined at step 475 can be compared to a threshold value to indirectly detect the presence of the analyte in the vial above the threshold and consequently detect DVE.

Further, because the reference signal is based on a substantially non-varying characteristic of the sensor, the isosbestic point, the ratio determined at step 475 is not affected by variability in components of optical blood culture measurement systems, temperature, or vial position.

Determination of the ratio at step 475 is one non-limiting example of normalizing a second fluorescence signal emitted in response to a second excitation wavelength in comparison to a first fluorescence signal emitted in response to a first excitation wavelength at an isosbestic point of the pH indicator. In certain embodiments, normalizing the second fluorescence signal can include calculating a ratio of the first fluorescence signal to the second fluorescence signal, calculating a ratio of the second fluorescence signal to the first fluorescence signal, subtracting the first fluorescence signal from second fluorescence signal, or subtracting the second fluorescence signal from the first fluorescence signal. In certain embodiments, normalizing the second fluorescence signal can include applying any function using an input based on the first fluorescence signal and an input based on the second fluorescence signal such that the variability in the result of the function is less than the combined variability of both fluorescence signal inputs. The variability in both fluorescence inputs can move in tandem, such as excitation intensity or fluorophore concentration, and can thus be canceled out or otherwise compensated for.

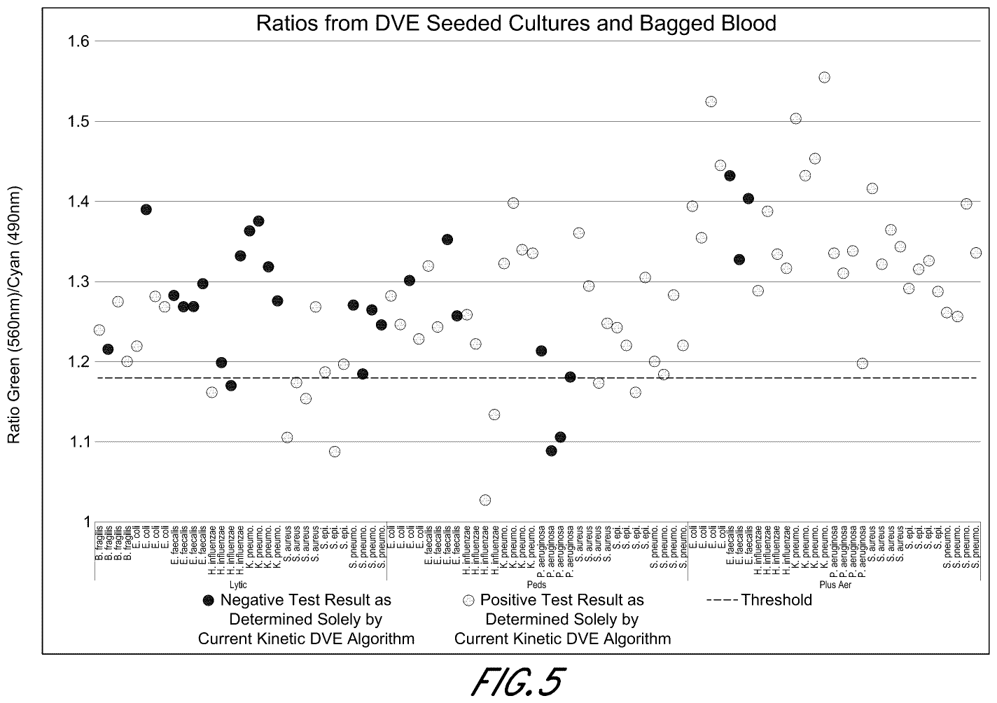

FIG. 5 shows the results of an experiment performed using the process 400. In the experiment, ninety-six (96) vials were provided with several media types. Bagged blood was introduced into the vials, and the vials were inoculated with organisms including several common strains of bacteria. In this experiment, the test vials were inoculated with Bacteroides fragilis, Escherichia coli, Enterococcus faecalis, Haemophilus influenzae, Klebsiella pneumoniae, Staphylococcus aureus, Staphylococcus epidermidis, Streptococcus pneumoniae, and Pseudomonas aeruginosa After inoculation, the vials were allowed to incubate for 24 hours before being placed in a measurement system, such as the measurement system 100. After 24 hours, the 96 bottles were loaded into the measurement system at a rate of 48 vials per day, and data was collected for 24 subsequent hours. In the measurement system, the test vials were excited with a cyan/blue light, and the intensities of resultant fluorescence signals emitted from the test vials were measured and recorded. About one minute after excitation with the cyan/blue light, the test vials were excited with green light, and the intensities of resultant fluorescence signals emitted from the test vials were measured and recorded. Independent excitation and measurement using each of the cyan/blue light and the green light was repeated about every 10 minutes during the data collection period.

Following the initial 24-hour incubation period, it is believed that each vial should have had sufficient bacterial growth to mimic a DVE scenario. A threshold for DVE detection was calculated empirically based on previous experimental data using fresh blood. As shown in FIG. 5, the threshold in this non-limiting example was 1.18.

In the graph shown in FIG. 5, the Y-axis represents a ratio as determined at step 475 of the process 400. Each circle on the graph represents one of the 96 vials. As shown in FIG. 5, emissions from eighty-four (84) of the vials resulted in ratios above the threshold value of 1.18 (illustrated in FIG. 5 as 84 circles located above the dashed 1.18 threshold line), indicating that the process 400 detected DVE for those vials, correlating to 87.5% sensitivity. Twelve (12) vials resulted in ratios below the threshold value (illustrated in FIG. 5 as 12 circles located below the dashed 1.18 threshold line), indicating that 12.5% of the test vials were deemed to be false negatives.

The graph shown in FIG. 5 also demonstrates detection of DVE using kinetic DVE algorithms. Each circle shown with a dotted interior represents a vial in which kinetic DVE algorithms detected DVE, and each circle shown with a filled interior represents a vial in which kinetic DVE algorithms failed to detect DVE. As shown, the kinetic DVE algorithms detected DVE in sixty-nine (69) vials, corresponding to 71.9% sensitivity. These results indicate a higher sensitivity to DVE events (greater incidence of DVE detection) using the process 400 in accordance with implementations of the present disclosure, in comparison to detection of DVE events using kinetic DVE algorithms alone.

Advantageously, the process 400 in accordance with implementations of the present disclosure can be used in combination with kinetic DVE algorithms to detect DVE in a vial to increase the overall rate at which DVE events are accurately detected. As shown in FIG. 5, for example, a combination of the process 400 and the DVE algorithms yields only 3 false negatives (shown in FIG. 5 as three (3) circles with filled interiors below the dashed 1.18 threshold line), corresponding to 96.9% sensitivity. Accordingly, detecting DVE events using a combination of current kinetic DVE algorithms and systems and methods according to the present disclosure can result in a substantial increase in sensitivity. In the non-limiting experiment corresponding to FIG. 5, sensitivity increased from 71.9% (27 of 96 vials not recognized as DVE events) to 96.9% (3 of 96 vials not recognized as DVE events).

Current methods of detecting DVE using kinetic DVE algorithms rely on detection of a change in signal over time. For example, measurements may be taken periodically (for example, every 10 minutes), and a rate of change can be determined. This rate of change may be proportional to a rate of change of pH in the test vial. In test vials in which DVE has not occurred, the rate of change may be initially low. In a test vial in which DVE has occurred, an initial measurement may be high. In such instances, all subsequent measurements will also be high, and kinetic DVE algorithms may detect a relatively low rate of change. Growth of bacteria or other microorganisms in a test vial is limited by the amount of nutrients in the vial. After the nutrients are exhausted, growth of bacteria or other microorganisms stops, which consequently stops the pH in the vial from changing further. If no rate of change (or a low rate of change) is detected, the current kinetic DVE algorithms will not determine DVE, potentially resulting in a false negative. In current practice, an operator may have to monitor a DVE measurements for a patient's test vial for 12 to 18 hours before deciding that tests are inconclusive. Using the systems and methods described herein, which do not rely on the pH in the test vial during an initial detector reading, an operator can immediately determine that DVE has occurred, and can proceed to obtain another blood sample from a patient for a new test rather than waiting 12-18 hours.

The use of a reference signal that is not dependent on a measured intensity of a fluorescence signal emitted from a test vial, such as the reference signals described with respect to FIGS. 2-4, can have numerous advantages over a reference signal that is dependent on an intensity of a fluorescence signal emitted from a test vial. Use of a reference signal in accordance with embodiments described herein can improve detector sensitivity by eliminating variability in an initial detector reading as a source of variability of the reported measurement system reading. Variability in an initial detector reading contributes proportionally to uncertainty in the measurement system output, which is equivalent to the resolution capability of the measurement system itself. In embodiments described herein in which the variability of the initial sensor reading to normalize detector readings is eliminated, the sensitivity of the detector is improved which advantageously results in a higher resolution measurement system.

Further, use of a reference signal in accordance with the embodiments described herein can improve or eliminate error due to DVE because the output signal of the measurement system is not normalized using an initial detector reading. Use of a reference signal in accordance with the embodiments described herein also obviates the need to compensate for sensor temperature fluctuations.

As described above, in certain embodiments detecting DVE events using a combination of kinetic DVE algorithms and systems and methods according to the present disclosure can result in a substantial increase in sensitivity. For example, in certain embodiments, a combination of a measured rate of change over time of a fluorescence output configured to change in response to proliferation of the analyte in the test device and a normalized measurement of intensity (for example, the ratio generated at step 475 of the process 400) normalized using a reference signal generated using an isosbestic point (for example, “Signal 1” at step 435 of the process 400 which does not change as a result of changes in pH of the pH indicator) can result in a substantial increase in sensitivity. In certain embodiments, the combination can be a summation of the measured rate of change over time of the fluorescence output configured to change in response to proliferation of the analyte in the test device and the normalized measurement of intensity normalized using the reference signal generated using the isosbestic point.

In certain embodiments, the systems and methods described herein can be used with other algorithms to reduce noise associated with measurements from a single (for example, green) channel. In certain embodiments, the systems and methods described herein can be used with other algorithms to detect a degradation in equipment of the measurement system, such an LED or a photodiode.

There are additional advantages associated with embodiments of the present disclosure. A reference signal that includes or is related to an isosbestic point of a component in the measurement system will not vary in its specific wavelength position for a given concentration of the component. This lack of variance in the reference signal makes it ideally suited to normalize detector readings of the measurement system. At the same time, a reference signal that does not vary based on a measured intensity of a fluorescence signal emitted from a test vial as described herein can serve as a real-time quality indicator for a measurement system, such as measurement system 100.

For example, because embodiments of the reference signals according to the present disclosure should be substantially constant or should not vary substantially during testing of a vial sample placed in a measurement system, the reference signal can be measured during the assay vial test duration to determine errors in the measurement system. In certain embodiments, the measurement system can be configured to disregard data if the reference signal varies during the assay vial test duration. Alternatively, the measurement system may be programmed to correct or accommodate sample output signals based on detected variation in the reference signals described herein.

In addition, the absolute level of an isosbestic signal reading from a measurement system can be useful to determine the health of the measurement system itself. The isosbestic signal will be expected to vary only based on the amount and/or concentration of fluorophore in the sensor and the bottle optical characteristics, the expected range of which can be determined through empirical testing or other suitable methods. An isosbestic signal that is below an established threshold could indicate either a bad bottle/sensor, or a degraded and/or failing measurement system. Identification of the isosbestic signal reading that is below the established threshold can alert the operator of the measurement system to check for and remedy these conditions.

Additionally, the use of a reference signal as described herein can allow for identification of a particular test vial or other test device. By design, reference signals in accordance with the present disclosure do not change between test devices. As a result, the reference signal may act as an identification marker that can be correlated with the test device and measured to confirm the identity thereof. This identification marker may be used as verification that a sensor-containing test device is supplied by a specific manufacturer. The identification marker may also serve as an indication that a competitor has copied a test device sensor chemistry. For example, a reference signal at an isosbestic point may be determined for a competitor test device and compared to known test device reference signals to determine that the competitor has copied a specific, known test device sensor chemistry.

Additional examples of non-varying reference signals and their use in optical blood culture measurement systems are described in U.S. Patent Application Publication No. 2021/0262936, which is hereby incorporated by reference in its entirety and for all purposes.

Implementations disclosed herein provide systems, methods and apparatus for optimizing detection of optical signals indicating the presence of an analyte of interest in a sample. One skilled in the art will recognize that these embodiments may be implemented in hardware, software, firmware, or any combination thereof.

In addition to the benefits described above, embodiments of the systems and methods described herein can be advantageously implemented without changing consumable components in current blood culture measurement systems. For example, implementations of the presently disclosed technology can be implemented without changing the assay bottle including its contents compromised of media, nutrient solution, and a sensor. Further, embodiments of the systems and methods described herein can be implemented with a one-time cost, which can be limited to the total cost of adding optical interrogation at specified wavelengths and a related algorithm code to process optical measurements and determine a reference signal as described herein. In certain embodiments, the light source that excites the pH indicator with a wavelength of light that is at or near the isosbestic point of the pH indicator (for example, the blue/cyan LED in the non-limiting examples described herein) can be optionally omitted from the detection process. Optional omission of this light source can allow users to use blood measurement systems implementing the presently disclosed technology in the same way as current blood culture measurement systems.

It will be further understood that the embodiments of the disclosed technology are not limited to blood culture measurement systems, and can be applied to other types of optical detection systems that rely on sensors or other materials that have a non-varying characteristic suitable for use in generating a reference signal for normalization of detector readings, such as an isosbestic point. For example, in certain embodiments, the disclosed technology can be applied to immunoassays, including immunoassays in which the immunoassay is monitoring output as a function of time and immunoassays in which the immunoassay is a one point in time test (i.e., an episodic test). When the immunoassay is an episodic test, a ratio of the test output to a reference signal generated using the isosbestic point can be used as an assay quality indicator or as surveillance method indicating that an immunoassay has been copied or counterfeited.

The functions described herein may be stored as one or more instructions on a processor-readable or computer-readable medium. The term “computer-readable medium” refers to any available medium that can be accessed by a computer or processor. By way of example, and not limitation, such a medium may comprise RAM, ROM, EEPROM, flash memory, CD-ROM or other optical disk storage, magnetic disk storage or other magnetic storage devices, or any other medium that can be used to store desired program code in the form of instructions or data structures and that can be accessed by a computer. Disk and disc, as used herein, includes compact disc (CD), laser disc, optical disc, digital versatile disc (DVD), floppy disk and Blu-ray® disc where disks usually reproduce data magnetically, while discs reproduce data optically with lasers. It should be noted that a computer-readable medium may be tangible and non-transitory. The term “computer-program product” refers to a computing device or processor in combination with code or instructions (e.g., a “program”) that may be executed, processed or computed by the computing device or processor. As used herein, the term “code” may refer to software, instructions, code or data that is/are executable by a computing device or processor.

Software or instructions may also be transmitted over a transmission medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of transmission medium.

The methods disclosed herein comprise one or more steps or actions for achieving the described method. The method steps and/or actions may be interchanged with one another without departing from the scope of the present disclosure. In other words, unless a specific order of steps or actions is required for proper operation of the method that is being described, the order and/or use of specific steps and/or actions may be modified without departing from the scope of the present disclosure.

The term “determining” encompasses a wide variety of actions and, therefore, “determining” can include calculating, computing, processing, deriving, investigating, looking up (e.g., looking up in a table, a database or another data structure), ascertaining and the like. Also, “determining” can include receiving (e.g., receiving information), accessing (e.g., accessing data in a memory) and the like. Also, “determining” can include resolving, selecting, choosing, establishing and the like.

The phrase “based on” does not mean “based only on,” unless expressly specified otherwise. In other words, the phrase “based on” describes both “based only on” and “based at least on.”

In the foregoing description, specific details are given to provide a thorough understanding of the examples. However, it will be understood by one of ordinary skill in the art that the examples may be practiced without these specific details. For example, electrical components/devices may be shown in block diagrams in order not to obscure the examples in unnecessary detail. In other instances, such components, other structures and techniques may be shown in detail to further explain the examples.

Headings are included herein for reference and to aid in locating various sections. These headings are not intended to limit the scope of the concepts described with respect thereto. Such concepts may have applicability throughout the entire specification.

It is also noted that the examples may be described as a process, which is depicted as a flowchart, a flow diagram, a finite state diagram, a structure diagram, or a block diagram. Although a flowchart may describe the operations as a sequential process, many of the operations can be performed in parallel, or concurrently, and the process can be repeated. In addition, the order of the operations may be re-arranged. A process is terminated when its operations are completed. A process may correspond to a method, a function, a procedure, a subroutine, a subprogram, etc. When a process corresponds to a software function, its termination corresponds to a return of the function to the calling function or the main function.

The previous description of the disclosed implementations is provided to enable any person skilled in the art to make or use embodiments of the present disclosure. Various modifications to these implementations will be readily apparent to those skilled in the art, and the generic principles defined herein may be applied to other implementations without departing from the spirit or scope of the present disclosure. Thus, the present disclosure is not intended to be limited to the implementations shown herein but is to be accorded the widest scope consistent with the principles and novel features disclosed herein.

What is claimed is:

1. A method of determining the presence of an analyte of interest in a blood sample within a test device comprising a sensor in aqueous media, the method comprising:
transmitting light to the test device at a first excitation wavelength at which a light absorption of the sensor remains substantially constant as a pH of the aqueous media changes;
measuring an intensity of a first fluorescence signal emitted from the sensor in the test device in response to the first excitation wavelength;
transmitting light to the test device at a second excitation wavelength, the second excitation wavelength being different from the first excitation wavelength;
measuring an intensity of a second fluorescence signal emitted from the sensor in the test device in response to the second excitation wavelength; and
normalizing, with the first fluorescence signal, the intensity of the second fluorescence signal emitted from the test device in response to the second excitation wavelength.

2. The method of claim 1, wherein normalizing the intensity of the second fluorescence signal comprises generating a ratio comparing the second fluorescence signal and the first fluorescence signal.

3. The method of claim 2, further comprising comparing the ratio to a threshold value.

4. The method of claim 3, further comprising determining the presence of the analyte when the ratio exceeds the threshold value.

5. The method of claim 1, wherein the first excitation wavelength is at an isosbestic point of a component of the sensor.

6. The method of claim 5, wherein the sensor comprises a pH indicator and a fluorophore, and wherein the isosbestic point is an isosbestic point of the pH indicator.

7. The method of claim 1, wherein the first excitation wavelength is in the blue/cyan range.

8. The method of claim 1, wherein the first excitation wavelength is between 485 nm and 495 nm.

9. The method of claim 1, wherein the first excitation wavelength is about 490 nm.

10. The method of claim 1, wherein the second excitation wavelength is in the green range.

11. The method of claim 1, wherein the second excitation wavelength is between 550 nm and 560 nm.

12. The method of claim 1, wherein the second excitation wavelength is about 555 nm.

13. The method of claim 1, further comprising:
measuring a rate of change over time of a fluorescence output configured to change in response to proliferation of the analyte in the test device; and
determining the presence of the analyte based on the normalized intensity of the second fluorescence signal and the measured rate of change over time of the fluorescence output configured to change in response to proliferation of the analyte in the test device.

14. The method of claim 13, wherein determining the presence of the analyte based on the normalized intensity of the second fluorescence signal and the measured rate of change over time of the fluorescence output configured to change in response to proliferation of the analyte in the test device comprises performing a summation of the normalized intensity of the second fluorescence signal and the measured rate of change over time of the fluorescence output configured to change in response to proliferation of the analyte in the test device.

15. A system for determining the presence of an analyte of interest in a blood sample, the system comprising:
a blood culture test device comprising a sensor in an aqueous media and configured to receive a blood sample;
a first light source;
a first excitation filter, wherein the first excitation filter is configured to filter light from the first light source to provide light to the sensor at a first excitation wavelength at which a light absorption of the sensor remains substantially constant as a pH of the aqueous media changes;
a second light source;
a second excitation filter, wherein the second excitation filter is configured to filter light from the second light source to provide light to the sensor at a second excitation wavelength, the second excitation wavelength being different from the first excitation wavelength;
one or more detectors configured to measure an intensity of a first fluorescence signal emitted from the sensor in the test device in response to the first excitation wavelength and an intensity of a second fluorescence signal emitted from the sensor in the test device in response to the second excitation wavelength; and
a processor configured to normalize the measurement of the intensity of the second fluorescence signal emitted from the sensor in the test device in response to the second excitation wavelength using the measurement of the intensity of the first fluorescence signal emitted from the sensor in the test device in response to the first excitation wavelength.

16. The system of claim 15, wherein the processor is configured to normalize the measurement of the intensity of the second fluorescence signal by generating a ratio comparing the second fluorescence signal and the first fluorescence signal.

17. The system of claim 16, wherein the processor is further configured to compare the ratio to a threshold value.

18. The system of claim 17, wherein the processor is further configured to determine the presence of the analyte when the ratio exceeds the threshold value.

19. The system of claim 15, wherein the first excitation wavelength is at an isosbestic point of a component of the sensor.

20. The system of claim 19, wherein the sensor comprises a pH indicator and a fluorophore, wherein the isosbestic point is an isosbestic point of the pH indicator.

21. The system of claim 15, wherein the first light source comprises a blue or cyan LED light source.

22. The system of claim 15, wherein the first excitation wavelength is between 485 nm and 495 nm.

23. The system of claim 15, wherein the first excitation wavelength is about 490 nm.

24. The system of claim 15, wherein the second light source comprises a green LED light source.

25. The system of claim 15, wherein the second excitation wavelength is between 550 nm and 560 nm.

26. The system of claim 15, wherein the second excitation wavelength is about 555 nm.

27. The system of claim 15, wherein the one or more detectors are configured to measure a rate of change over time of a fluorescence output configured to change in response to proliferation of the analyte in the test device; and
wherein the processor is configured to determine the presence of the analyte based on the normalized measurement of the intensity of the second fluorescence signal and the measured rate of change over time of the fluorescence output configured to change in response to proliferation of the analyte in the test device.

28. The system of claim 27, wherein in the processor is configured to determine the presence of the analyte based on a summation of the normalized measurement of the intensity of the second fluorescence signal and the measured rate of change over time of the fluorescence output configured to change in response to proliferation of the analyte in the test device.
